(19) United States
(12) Patent Application Publication
(10) Pub. No.: US 2003/0170730 A1
(43) Pub. Date: Sep. 11, 2003

(54) Assay method, peptides and reagent kits for testing mrp-specific inhibitors

(76) Inventors: Eva Bakos, Budapest (HU); Balazs Sarkadi, Budapest (HU); Andras Varadi, Budapest (HU); Gabor Tusnadi, Budapest (HU); Raymond Evers, East Brunswick, NJ (US)

Correspondence Address: Keil & Weinkauf, 1350 Connecticut Avenue NW, Washington, DC, 20036, US

(21) Appl. No.: 10/343,013

(22) PCT Filed: Jul. 30, 2001
(86) PCT No.: PCT/HU01/00085

(30) Foreign Application Priority Data
Jul. 28, 2000 (HU) P0002996

(51) Int. Cl. G01N 33/53; C07K 16/18

(52) U.S. Cl. 435/7.1; 530/388.1

(57) ABSTRACT

The invention relates to a method for testing L₀-specific inhibitors of transporter proteins of the MRP-family and for testing inhibitors specific to the predicted amphipatic helical region of the L₀ region, the tested inhibitors L₀ peptide, mutants thereof, polynucleotides coding the peptides. Vectors and hosts cells comprising the polynucleotides and reagent kits for testing and developing the inhibitors. The invention is especially useful for combatting multidrug resistance in cancer patients and in the field of related research.

The invention relates to an assay method for testing L₀-specific inhibitors of transporter proteins of the MRP-family, L₀ peptide and reagent kits for testing and developing the inhibitors and L₀-specific inhibitors of the MRP-family provided by using the method or reagent kit of the invention.

The invention is especially useful in the field of multidrug resistance research and cancer treatment.

MDR1 (P-glycoprotein) and MRP1 (multidrug; resistance protein 1) are members of the ABC (ATP-Binding Cassette) transporter family that are able to extrude chemotherapeutic drugs from tumor cells in an ATP-dependent manner. Whereas MDR1 preferentially transports hydrophobic, slightly positive molecules, MRP1 transports glutathione conjugates (e.g. leukotriene C₄) and other large hydrophobic molecules conjugated to negatively charged ligands like sulfate and glucuronide [Gottesman, M. M. and Pastan, J. (1993) Annu. Rev. Biochem. 62, 385-427; McGrath, T., Latoud, C., Arnold, S. T., Safa, A. R., Felsted, E. R., and Center, M. S. (1989) Biochem. Pharmacol. 38, 3611-3619; Muller, M., Meijer, C., Zaman, G. J. R., Borst, P., Scheper, R. J., Mulder, N. H., de Vries, E. G. E., and Jansen, P. L. M. (1994) Proc. Natl. Acad. Sci. USA 91, 13033-13037; Jedlitschky, G., Leier, I., Buchholz, U., Center, M. S., and Keppler, D. (1994) Cancer Res. 54 4833-4836; Jedlitschky, G., Leier, I., Buchholz, U., Barnouin, K., Kurz, G., and Keppler, D. (1996) Cancer Res. 56, 988-994; Loe, D. W., Alrnquist, K. C., Cole, S. P. C., and Deeley, R G. (1996) J. Biol. Chem. 271, 9683-9689]. MRP1 also transports unmodified neutral and basic organic compounds, probably by a co-transport mechanism together with glutathione [Loe, D. W., Alrnquist, K. C., Cole, S. P. C., and Deeley, R. G. (1996) J. Biol. Chem. 271, 9675-9682; Loe, D. W., Deeley, R. G., and Cole, S. P. C. (1998) Cancer Res. 58, 5130-5136; Evers, r., de Haas, M., Sparidans, R., Bijnen J., Wiclenga, P. R., Lankelma, J., and Borst, P. Br. J. Cancer, in press]. MDR1 Pgp and MRP1 seem to be major players in chemotherapy resistance in numerous forms of cancer, although homologs of these two transporters, as well as the recently identified breast cancer resistance protein (BCRP or, in other name, ABCG2) may also be involved in this clinical phenomenon [Doyle, L. A., Yang, W., Abruzzo, L. V., Krogmann, T., Gao, Y., Rishi, A. K., and Rioss, D. D. (1998) Proc. Natl. Acad. Sci. USA; 95 15665-15670].

MRP1 has been shown to be a high affinity primary active transporter for the glutathione-conjugated eicosanoid, leukotriene C₄ (LTC₄) [Leier, I., Jeditschky, G., Buchholz, U., Cole, S. P. C., Deeley, R. C., and Keppler, D. (1994) J. Biol. Chem., 969, 27807-27810 and Muller, M., (1994), see above] and to transport various other compounds that are conjugated to glutathione, sulfate or glucuronide [Leier, I., (1994), Jedlitschky, G., (1996), Loe D. W., (1996), Zaman, G. J. R., Cnubben. N. H. P., van Bladeren, P. J., Evers, R., and Borst, P., (1996) K B S Lett., 391, 125-130], The physiological functions of MRP1 range from the mediation of an inflammatory response to the elimination of certain xenobiotics [Holló, Zs., Homolya L., Hegedús, T., and Sarkadi B. (1996) FEBS Lett., 383, 99-104; Grant, C. E., Valdimarsson, G., Hipfner, D. R., Alrnquist, K. C., Cole, S. P. C., and Deeley, R. G., (1994) Cancer Res., 51, 357-361; Wijnholds, J., Evers, R. van Leusden, M. R., Mol. C. A., Zaman G. J. Mayer, U., Reijnen, J. M., van der Valk, M., Krimpenfort, P., and Borst, P. (1997), Nat. Med. 3, 1275-1279; Lorico, A., Rappa G., Finch, R. A., Yand. D., Flavell, R. A. and Sarcorelli, A. C., (1997) Cancer Res. 57, 5238-5242; Rappa G., Lorico, A., Flavell. R. A. and Sartorella. A. C., (1997), Cancer Res., 57, 5232-5237] and this protein may play a role in the chemotherapy resistance of several types of cancer.

Vanadate inhibits ATP-dependent drug transport both by MDR1 and MRP1 [Scarborough G. A. (1995) J. Bioenerg Biomembr. 27, 3741; Bakos, É., Evers, R., Sinkó, E., Váradi, A., Borst, P., and Sarkadi, B. (2000) Mol. Pharmacol. 57, 760-768; Mao, Q., Leslie, E. M., Deeley, R. G., and Cole, S. P. (1999) Biochim Biophys Acta 1461 69-82; Chang, X. B., Hou, Y. X., and Riordan, J. R. (1998) J. Biol. Chem. 273, 23844-23848] and in the presence of vanadate, the trapping of an adenine nucleotide in these proteins has been demonstrated [Urbatsch, I. L., Sankaran, R., Weber, J. and Senior, A. E. (1995), J. Biol. Chem., 270, 19383-19390; Taguchi, Y., Yoshida, A., Takada, Y., Komano, T., and Ueda, K. (1997) FEBS Lett. 401, 11-14]. Transported compounds specifically increase the rate of vanadate-dependent nucleotide trapping in MRP1 [Taguchy Y et al., FEBS Lett. 401, 11-14 (1997)], similarly to what has been shown for hydrophogic drugs in the case of MDR1 Pgp [Szabó, K. et al, J. Biol. Chem 273, 10132-10138 (1998)]. Vanadate dependent drug stimulated nucleotide trapping reflects a partial reaction of the multidrug transporters and thus can be used to examine their functional characteristics.

Other inhibitors of MRP1 are also known in the art.

One of them is PAK-105P which efficiently increased CPT-11- and SN-38-accumulation in C-A120- and KB/MRP cells in a 10 nM concentration [Chen. et al., Mol. Pharmacol. 55(5), 921-8 (1999)].

Draper et al [Br. J. Cancer 75(6), 810-15 (1997)] studied the effect of indomethacine, a known inhibitor of cyclooxigenase and glutathione-S-transferase and a modulator of anion transport, on murine and human cells expressing MRP and found that the indomethacine effectively inhibited the efflux of a fluorescent dye, BCECF and increased its accumulation. Further measurements and a comparision with the MDR1 protein suggested that the inhibition is MRP1-specific; however, the question that whether indomethacine acts via the inhibition of glutathione-S-transferase or competes directly with the transported drug at the transport site, could not be decided.

The problem that a decrease in drug resistance is not the result of the potential inhibitor's effect on MRP1, is mentioned also in the work of Curtin and Turner [Eur. J. Cancer 35(6), 1020-5 (1999)]. The authors studied the sensitizing effect of an inhibitor of nucleoside transport, dipiridamol (DP) on cells overexpressing MRP1 and found that sensitization is rather due to a decrease in the cellular GSH level than a modification in MRP1 mediated drug efflux. Versantvoort et al. [Br. J. Cancer 72(1) 82-9 (1995)] came to a similar conclusion upon studying buthyonine-sulfoxide, an inhibitor of glutathione synthesis.

Said problem may emerge in case of any test which is aimed to detect MRP1 activity indirectly, in living cells, via drug efflux or accumulation. In the state of art, however, most often such tests are applied.

One of the most well-known MRP1 inhibitor is MK-571, a leukotriene-D4-receptor antagonist. Van der Kolk et al [Clin. Cancer Res. 4(7) 1727-36 (1998)] worked out a functional assay for measuring MRP1 activity, which is based on carboxyfluorecsein (CF) transport by MRP1 and its inhibition by MK-571. This method might turn out to be suitable for testing the effect of other MRP1 inhibitors as well, though this possibility is not mentioned by the authors.

The inhibitors of the art were not developed by purposeful designs, thus, their mechanism of action is not known. Moreover, they probably influence other biochemical pathways of the organism, as well. Therefore, there is an existing need for an assay method or test system by which a potential inhibitor can be tested for definite MRP functions.

For that purpose in proteins of the MRP family the key structural region, for which the inhibitor is specific, should be distinguished functionally, or its functions should be detectable so that an inhibitor specific for this particular, important structural region may be tested.

However, no test, suitable for detecting functions associated with various regions of the molecule has been known so far.

To provide such a test, information is required on functions and interactions of individual regions or domains of this large molecule characterized by complicated domain-domain interactions.

Relevant data of the art in respect of functions and “co-operation” of some significant structural units of MRP1 are briefly summarized below.

MDR1 and MRP1 share a similar core structure, containing a tandem repeat of transmembrane domains (TMDs) and cytoplasmic ATP-binding cassette (ABC) regions. However, MRP1 and several homologues form a subfamily of ABC-transporters, characterized by a triple membrane-bound domain structure. These include an MDR1-like core region, containing two ABC units and two membrane-bound domains (TMD₁ and TMD₂), and an N-terminal polypeptide region of about 280 amino acids, forming a cytoplasmic loop and a membrane-bound domain (L₀ and TMD₀, respectively, see FIG. 1A; [see the FIG. 1.a, and Tusnády, G. E.; Bakos, É., Váradi, A., and Sarkadi, B. (1997) FEBS Lett. 402, 1-3 and Bakos, É., Evers, R., Szakács, G., Tusnády, G. E., Welker, E., Szabó, K., de Haas, M., van Deemter, L., Borst, P., Váradi, A., and Sarkadi, B. (1998) J. Biol. Chem. 273, 32161-32175]).

Intact whole MRP1 was expressed in baculovirus infected insect cells [Bakos, É., Hegedús, T., Holló, Zs., Welker, E., Tusnády, G. E., Zaman, G. J. R., Flens, M. J., Váradi, A., and Sarkadi, B. (1996) J. Biol Chem. 271, 12322-12326; Gao, M., Loe, D. W., Grant, C. E., Cole, S. P. C. and Deeley, R. G. (1996), J. Biol. Chem., 271, 27782-27787; and Gao, M., Yamazaki, M., Loe, D. W, Westlake, C. J., Grant, C. E., Cole, S. P. C., and Deeley, R. G. (1998) J. Biol. Chem. 273, 10733-10740] and, though the protein proved to be underglycosylated in this expression system, its basic structural and transport properties were the identical with those found in mammalian cells [Gao, M et al, (1996), see above]. Further, MRP1 mutants were expressed in insect cells, which mutants did not contain the N-terminal TMD₀L₀ region of the protein and which were not active, while if the N-terminal TMD₀L₀ region was coexpressed with the C-terminal core region an functional LTC₄ transporter was obtained [Gao, M et al, (1996), see above].

In polarised cells MDR1 is routed to the apical membrane, while MRP1 to the basolateral membrane [Bakos, É., Evers, R., Szakács, G., Tusnády, G. E., Welker, E., Szabó, K., de Haas, M., van Deemter, L., Borst, P., Váradi, A., and Sarkadi, B. (1998) J. Biol. Chem. 273 32161-32175; Evers, R., Zaman, G. J., van Deemter, L., Jansen, H., Calafat, J., Oomen, L. C., Oude Elferink, R. P., Borst, P., and Schinkel, A. H. (1996) J. Clin. Invest. 97, 1211-1218]. In these publications, just prior to the elaboration of the invention, the role of N-terminal regions of MRP1 and delivery of human MRP1 to plasmamembrane were studied. It was shown that the MDR like core region has practically no transport activity and nucleotide occlusion activity [(Bakos et al (1998), see above]. However, transport function could be restored by co-expressing the whole N-terminal TMD₀L₀ region and the MDR1-like core region [see also Gao et al (1998), like above].

Further, carrying out experiments with mutants Bakos, É. and co-workers found that mutant MRP1 comprising the core region and L₀ region but not comprising the TMD₀ domain proved to be an active transporter molecule with a correct, i.e. basolateral membrane routing [Bakos et al, (1998), see above]. This earlier study suggested that the presence of L₀ region in the molecule is required to MRP1 mediated transport activity and basolateral membrane routing of the protein, in polarised cells.

However, certain confusion or ambiquity existed in the prior art regarding the role of TMD₀ region and the possible importance of the L₀ region itself to the molecular activity.

Namely, Gao et al [Gao et al (1998), see above] found that removal of the whole TMD₀ domain or only its first N-terminal transmembrane helix abolished MRP1 function (when removing the whole TMD₀ region Gao et al removed a very short part of the L₀ region as defined in the description). This led these authors to conclude that the entire TMD₀L₀ region is essential for transport activity. This is clearly contrary to the above results leading to the assumption that the L₀ΔMRP protein is fully functional. The above results strongly suggest that the intact character of functions of some of the molecule's structural regions (i.e. whether the said function is retained or not) highly depends on the molecular environment and on interactions with other parts or regions of the molecule. It could not be expected therefore that L₀ forms a distinguishable structural unit and, as such, can be studied in itself and also that other structural units of the molecule can be studied in themselfes.

In the state of art there is no definite suggestion regarding the role of L₀. Earlier it was considered as a cytoplasmatic (or sometimes even extracellular) loop, which only links (hence its name: linker region) the two large structural unit, the core region and the N-terminal transmembrane domain. Furthermore, no definite structural feature have been assigned to the L₀ region.

At first, aiming at elaborating the invention, present inventors also supposed that in order to develop a method or test system for testing an L₀ region specific inhibitor the L₀ region has to be expressed in the form of a chimeric protein, covalently linked to a protein (e.g. MDR) or a domain homologous but functionally unrelated to natural structural units of the MRP. The inventors hoped that thereby the significance of the L₀ region can be clarified. These experiments are described below.

Unexpectedly, it was found that if L₀ region is used, preferably expressed, as a separate molecule (L₀ peptide), optionally co-expressed with other MRP regions, its functions can be detected or assayed specifically and studied by themselfes; furthermore, novel functions can be found. Thus, the L₀ peptide can be a suitable tool for testing inhibitors.

Furthermore, surprisingly it was found that a shorter segment of the L₀ region has especially significant in respect of the functional character of the L₀ peptide and the whole transporter protein, and if said segment is neutralized or disabled, most of the L₀-functions (or MRP-functions) are damaged.

Thus, an object of the invention is to provide a method suitable for testing L₀ region specific inhibitors of transporter proteins of the MRP-family.

An other object of the invention is to provide a smaller segment within the L₀ region, which segment is itself important to the activity (operability) of the molecule.

A further object of the invention is to provide tools, e.g. peptides, recombinant constructs, expression vectors, suitable host cells and reagent kits useful in testing the inhibitors and in study of transporter proteins of the MRP family, e.g. in research of multidrug resistance.

Thus, the invention relates to an assay method for testing L₀-specific inhibitors of transporter proteins of the MRP family, comprising assaying an L₀ peptide exposed to the effect of a potential inhibitor, to determine whether at least one L₀-function was retained or at least partially lost. Preferably, the L₀ peptide is expressed and optionally the L₀ peptide is exposed to the effect of the potential inhibitor before the final formation of its native structure (processing).

According to a further preferred embodiment assaying the L₀ peptide is carried out by using a deletion mutant protein comprising an MRP-family transporter protein core region or its fragment capable of functioning and assaying at least one L₀-function manifesting itself only upon contact with the core region. Preferably, both the L₀ peptide and the core region are prepared by expression, or optionally by coexpression.

As an L₀-function e.g. the following functions can be assayed or determined: membrane binding, rescuing the core region of the protein to the physiological membrane compartment and/or restoring transporter protein function. Preferably, when the question is whether the transporter protein function is restored or not by the L₀ peptide, it is also determined whether at least one of the following functions of the whole transporter protein was retained or at least partially lost: nucleotide trapping in membrane preparations; membrane routing, preferably to a specific membrane area of polarised cells; ATP-ase activity; membrane transport of substrates, preferably substrate uptake in inside-out vesicles; vectorial membrane transport into or from polarised cells.

The invention further relates to an assay method for screening potential inhibitors of MRP family transporter protein specific for a segment of the L₀ region predicted to be an amphipatic helix comprising determining whether the potential inhibitor exhibits affinity for the segment predicted to be an amphipatic helix.

In a further preferred embodiment the invention relates to an assay method for testing inhibitors of MRP family transporter proteins specific for an L₀ region segment predicted to be an amphipatic helix, comprising assaying a transporter protein of the MRP family having exposed to the effect of a potential inhibitor; a functional transporter protein fragment comprising L₀ region; or an L₀ peptide and a protein consisting of a transporter protein core region (or a functional fragment thereof) said peptide and said protein being not linked covalently;

a) in respect of specific membrane routing and/or glycosilation of the protein or its fragment, and

b) in respect of any other function, preferably transporting capability of the protein, membrane binding of the L₀ peptide and/or MRP1 function restoring capability of the L₀ peptide, and

if the assayed functions of a) are retained and if the assayed functions of b) are damaged considering the result as the selective inhibiting effect of the inhibitor specific for the segment predicted to be an amphipatic helix.

In a further aspect the invention relates to an L₀ peptide or a mutant or homologue thereof at least partly at least one L₀-function of which is retained for use in testing, designing or developing L₀-specific inhibitors of transporter proteins of the MRP family, in multidrug resistance research or as a validated drug target. The peptide or its mutant comprises preferably amino acids of the amphipatic helix of the L₀ peptide of MRP1 or a part thereof, e.g. the segment comprising amino acids 216 to 227 or a part thereof, e.g amino acids 221 to 227, 220 to 228, 219 to 230 or 223 to 232.

Furthermore, the invention relates to a mutant L₀ peptide, carrying a mutation in the segment of the peptide predicted to be an amphipatic helix, preferably in segment 221 to 227, said mutation being preferably a deletion or a substitution.

The invention relates to a polynucleotide comprising a sequence coding for L₀ region of a transporter protein of the MRP family for use in expressing L₀ peptide. The polynucleotide can be an expression vector, preferably an expression vector capable of functioning in a bacterial, an insect and/or a mammalian cell. The invention further relates to host cells carrying a vector of the invention.

The invention further relates to reagent kits for developing or testing specific inhibitors of transporter proteins of the MRP family or for multidrug resistance research, said kit comprising a peptide, a polynucleotide or a host cell of the invention, and optionally further comprising an expression vector suitable to express the transporter protein or a functional mutant thereof, preferably a deletion mutant substantially consisting of the core region of the transporter protein, and/or host cells, preferably bacterial, insect and/or mammalian cells, in which the expression vector is operable, and/or reagents and buffers for the expression and/or assaying functions of the expressed L₀ peptide. Preferably, in reagent kits according to the invention the vector is a vector capable of functioning in E. coli, Sf9 and/or MDCKII cells and the host cells are E. coli, Sf9 and/or MDCKII cells.

Using the assay methods of the invention inhibitors can be obtained. Thus, the invention relates to drugs behaving as an L₀-specific inhibitor of a protein of the MRP family if assayed by a method of the invention. Preferably, said drug is capable to effect as an inhibitor due to being contacted with an L₀ region segment predicted to be an amphipatic helix or a part thereof.

### DEFINITIONS

A “Transporter protein of the MRP family” is a transporter protein having a domain structure of any of MRP1 to 6, preferably MRP1; in particular, according our present knowledge about domain structure, a transporter protein having a core region, an L₀-like, typical cytoplasmatic loop region and, in many cases but not necessarily, an N-terminal TMD₀ region. Such a protein is e.g. the SUR protein. Most recently, according to the HGNC gene family nomenclature, the MRP family is also called ABCC family. Thus, in an other aspect, the MRP family comprises also those transporter proteins which are classed as members of the ABCC family, e.g. upon genomic sequencing.

“Core region” of a protein of the MRP family is meant as the MDR-like core region of the protein, in particular a region comprising two ATP-binding casette (ABC) and two membrane bound domain (TMD₁ and TMD₂ domains).

“Corresponding regions” of proteins of the same family are meant as sequence regions proved to be homologous upon sequence alignment based on homology.

Herein, a “function” of any protein or its fragment is meant as any capability, suitability or non-structural feature or proper which is also characteristic of a variant having a sequence identical with the wild type. A “function” can appear either in vivo or in vitro. Preferably the function is an (at least in principle) detectable feature or property manifesting itself upon functioning. In this sense functioning is considered as any response of the protein or its fragment to an environmental effect during its whole life period from its synthesis to degradation. Thus, without any limitation, “functions” are e.g. the following: capability of the protein or its fragment to be glycosilized or folded properly, its targeting, assembly of the protein or participation of a fragment in such an assembly, activity or partial activity. Thus the terms “L₀-fuction”, “MRP1-function” are used in this sense.

Functions of proteins of the MRP family are e.g. the following:

The term “activity” of MRP comprises e.g. transport of a drug through the membrane carrying the MRP protein, ATP-ase activity etc. In a broad sense “activity” is meant as any partial reaction (e.g. substrate binding) of the whole reaction cycle of the enzyme as well as a partially damaged activity, e.g. nucleotide occlusion (trapping).

A further MRP-function is “routing” to a given membrane area. A function can be a form of a general function manifested in a particular environment of assay method, e.g. uptake of transported drugs into membrane vesicules comprising MRP protein or their transport through a membrane of polarized cells.

A fragment of a protein, e.g. of the MRP family, may show typical functions, e.g. membrane binding; resale of the core region by the L₀ peptide or by the N-terminal region to the plasmamembrane (or a specific membrane compartment); or restoration of the activity of the MRP protein.

Herein, the meaning of a “fragment capable of functioning” or a “functional fragment” is a fragment of a region with definite functions in a protein, a homologue or a mutant of said fragment at least one function of which is detectable.

“L₀ peptide” is a peptide molecule the amino acid sequence of which is identical with or a fragment, a homologue or a mutant of the amino acid sequence of the L₀ region of any MRP family proteins. Preferably, L₀ peptide is an L₀ region fragment capable of functioning. Preferably, the L₀ peptide is homologous to the sequence of the MRP1 204 to 281 amino acids, however, it should be understood that it may contain either more or less amino acids.

The “segment predicted to be an amphipatic helix” of the L₀ peptide is a segment predicted to be an amphipatic helix by the helical wheel prediction method or a corresponding region. According to our results this region plays a role in the membrane binding of the L₀ region of MRP family transporter proteins. More closely, based on sequence homology, a “segment predicted to be an amphipatic helix” is a conservative region corresponding to the about 220 to 232 amino acids of MRP1 or the helical part of this segment or a region comprising a part of this segment having features typical to said region.

### SHORT DESCRIPTION OF THE DRAWINGS

FIG. 1. Schematic representation (Panel A) and immunoblot detection of the MRP1-MDR1 chimeric proteins expressed in Sf9 cells (Panel B) and in MDCKII cells (Panels C and D)

Panel A. Schematic representation of the MRP1-MDR1 chimera proteins.

Panel B. Western blot analysis of Sf9 cell membranes containing MDR1, L₀MDR1, TMD₀MDR1, TMD₀L₀MDR1, or MRP1. Isolated membrane of Sf9 cells (5 μg) were subjected to electrophoresis in a 6% Laemmli-type gel, and immunoblotted as described in the Materials and Methods section. Lanes 1-5 were stained with the polyclonal antibody 4077, specific for human MDR1, while lanes 6-10 were developed with mAb R1, specific for human MRP1. Protein-antibody interactions were visualized using the enhanced chemiluminescence technique.

Panel C. Western blot with lysates from MDCKII wild-type cells and cells producing L₀MDR1, MDR1 or MRP1. 20 μg of protein was size fractionated in a denaturing 7.5% polyacrylamide gel. After electroblotting blots were incubated with either C219, recognising human MDR1 (lanes 1-4) or mAb R1 (lanes 5-8). The identity of the cell lines is indicated over the lanes. Panel D. Western blots showing MDCKII-MDR1 cells (lanes 1-4) and MDCKII-L₀MDR1 cells (lanes 5-8) treated with tunicamycin (3 μg/ml) for 0, 12, 25 or 40 h. MDR1 was visualized with mAb C219, and L₀MDR1 with mAb R1.

FIG. 2. LTC₄ transport (Panel A) and nucleotide trapping (Panel B) in Sf9 cell membranes containing MRP1-MDR1 chimeric proteins

Panel A. Time-course of ATP-dependent LTC₄ uptake in Sf9 membrane vesicles, expressing MRP1, MDR1, TMD₀MDR1, TMD₀L₀MDR1, L₀MDR1, or β-galactosidase. Membrane preparations were incubated with 50 nM LTC₄ at 23° C. and ATP-dependent uptake was calculated by subtracting the values obtained in the presence of 4 mM AMP from those in the presence of 4 mM ATP. Samples were taken at the time points indicated.

Panel B. Vanadate-dependent nucleotide trapping by MRP1-MDR1 chimeric proteins. Isolated membranes from Sf9 cells expressing MRP1 (lanes 1-3), MDR1 (lanes 4-6), L₀MDR1 (lanes 7-9), TMD₀MDR1 (lanes 10-12), and TMD₀L₀MDR1 (lanes 13-15) were labeled with Mg-8-azido-[α-³²P]-ATP in the presence of vanadate, as described in the Materials and Methods section [see also Szabó, K., et al, (1998), above]. The reaction buffer contained 30 μM verapamil in experiments documented in lanes 2, 5, 8, 11 and 14, and 600 nM LTC₄ in lanes 3, 6, 9, 12, and 15.

FIG. 3. Localization and transport properties of the chimera L₀MDR1 in MDCKII cells

Panel A. Immunolocalization of L₀MDR1 in MDCKII monolayers by confocal laser scanning microscopy. L₀MDR1 was detected by indirect immunofluorescence with mAb MRK16 (green signal, light signal on the figures). The upper part shows a top view of the monolayer, the lower part a vertical X/Z section. The arrow head indicates the position where the section was made. Nucleic acids were counterstained with propidium iodide (red signal).

Panel B. Transport of [³H]vinblastine by MDCKII-derived monolayers. Transport of vinblastine by MDCKII, MDCKII-MDR1 and MDCKII-L₀MDR1 cells is shown. Transport is presented as the fraction of total radioactivity added at the beginning of the experiment (t=0), appearing in the opposite compartment. PSC833 (0.1 μM) was present in both compartments to inhibit the endogenously present MDR1 Pgp present in these cells [Bakos É. et al, (1998), see above]. Squares: translocation from apical to basolateral. Circles: translocation from basolateral to apical. The experiments was performed in duplicate and repeated twice.

FIG. 4. Detection and function of MRP1 variants in Sf9 cell membranes

Panel A Schematic representation of the MRP1 protein and mutant proteins obtained from MRP1 by deletion.

Panel B: Western blot analysis of Sf9 cell membranes prepared from cells expressing MRP1 (lanes 1 and 5), ΔMRP1 (lanes 2 and 6), ΔMRP1+L₀ (lanes 3 and 7) or L₀ (lanes 4 and 8). Isolated membranes of Sf9 cells (5 μg) were subjected to electrophoresis in a 4-20% Laemmli-type gel, and immunoblotted. Blots were incubated with mAb R1 and M6, respectively,

Panels C and D: Time-course of ATP-dependent LTC₄ (Panel C) and NEM-GS (Panel D) uptake in Sf9 cell membrane vesicles containing MRP1, ΔMRP1, ΔMRP1+L₀, or β-galactosidase. Panel B: Membranes were incubated with 50 nM LTC₄ at 23° C.; Panel C: Membranes were incubated with 50 μM NEM-GS at 37° C. ATP-dependent uptake was calculated by subtracting the values obtained in the presence of 4 mM AMP from those in the presence of 4 mM ATP. Samples were taken at the time points indicated.

FIG. 5. Vanadate-dependent nucleotide trapping by MRP1 variants

Isolated membranes from Sf9 cells expressing MRP1 (lanes 1-3), ΔMRP1+L₀ (lanes 4-6), or ΔMRP1 (lanes 7-9), were labeled with Mg-8-azido-[α-³²P]-ATP in the presence of vanadate as described in the Materials and Methods section [see also Szabó, K. et al (1998), above]. The reaction buffer contained 600 nM LTC₄ (lanes 2, 5, and 8), or 560 μM NEM (lanes 3, 6, and 9).

FIG. 6. Immunolocalization of L₀ co-expressed with ΔMRP1 in MDCKII monolayers by confocal laser scanner microscopy

L₀ was detected by indirect immunofluorescence with mAb R1 (green color, light signal in the lateral membrane). The upper part shows a top view of the monolayer, the lower part a vertical X/Z section. The arrow head indicates the position where the section was made. Nucleic acids were counterstained with propidium iodide (red signal).

FIG. 7. Membrane attachment of the L₀ region of MRP1 expressed in Sf9 cells

Panel A: Detection of the L₀ region of human MRP1, expressed in Sf9 cells by Western blotting. Samples were fractionated in a 20% Laemmli-type gel immunoblotted and incubated with mAb R1. C: cell extract (5 μg); M: membrane fraction (5 μg); CP: cytoplasmic fraction, representing equivalent volumes of the cytoplasma as compared to membrane fractions.

Panel B: Effects of KCl (1 M, lanes 2, 3), Urea (5M, lanes 4, 5) or Triton X-100 (1%, lanes 6, 7) on the membrane attachment of the L₀ region of MRP1. Membrane samples were washed with the respective agents and then size fractionated as in Panel A. Lane 1: isolated Sf9 cell membranes, Lanes 2-7: membranes washed with the respective agents. P: membrane pellet fraction; S: supernatant fraction. Samples represent equivalent initial volumes of each fraction.

Panel C: Computer-predicted schematic representation of an amphipathic helix within the L₀ region of various MRP proteins. Helical wheel plot of residues 221-233 in MRP1, 216-228 in MRP2, 220-232 in MRP3, 24-36 in MRP4, 112-124 in MRP5, and 216-228 in MRP6. Hydrophobic residues are circled.

FIG. 8. Characterization of MRP1Δ(223-232) and L₀Δ(223-232) in Sf9 cells

Panel A: Western blot analysis of Sf9 cell membranes containing MRP1 or MRP1Δ(223-232). Isolated membranes of Sf9 cells (5 μg) were subjected to electrophoresis in a 6% Laemmli-type gel, and immunoblotted as described in the Materials and Methods section, with monoclonal antibody R1, specific for human MRP1.

Panel B: Time-course of ATP-dependent LTC₄ uptake in Sf9 cell membrane vesicles, expressing MRP1, MRP1Δ(223-232), or β-galactosidase. Membrane preparations were incubated with 50 nM LTC₄ at 23° C. and ATP-dependent uptake was calculated by subtracting the values obtained in the presence of 4 mM AMP from those in the presence of 4 mM ATP. Samples were taken at the times indicated.

Panel C: Detection of the L₀Δ(223-232) mutant region of human MRP1, expressed in Sf9 cells. Samples were fractionated on a 20% Laemmli-type gel, and immunoblotted by R1 antibody as described in the Materials and Methods section. C: cell extract (5 μg); M: membrane fraction (5 μg); CP: cytoplasmic fraction, representing an equivalent initial volume compared to the total membrane fraction.

FIG. 9. Characterization of MRP1Δ(223-232) in MDCKII cells

Panel A. Western blot with lysates from MDCKII wild-type, MDCKII-MRP1 and MDCKII-MRP1Δ(223-232) cells. Four or 20 μg of protein were size fractionated. The blot was incubated with mAb R1. The identity of the cell lines is indicated over the lanes.

Panel B. Western blot with lysates from MDCKII-MRP1 and MDCKII-MRP1Δ(223-232) cells, treated with tunicamycin (3 μg/ml) for 0, 12, 25 or 40 h. The blots were incubated with mAb R1.

Panel C. Immunolocalization of MRP1Δ(223-232) in MDCKII monolayers. MRP1Δ(223-232) was detected as in FIG. 6 with mAb R1.

Panel D. Transport of DNP-GS by MDCKII derived monolayers. Cells were incubated with [¹⁴C]CDNB (2 μM) in both the apical and basal compartment. This hydrophobic compound diffuses over the plasma membrane and intracellularly reacts to glutathione by the mediation of glutathione S-transferases. Samples were taken at t=0, 1, 3, 6, and 20 min from both compartments and extracted with ethyl acetate to separate free CDNB from intracellularly formed DNP-GS. Squares: transport to the basolateral compartment. Circles: transport to the apical compartment. The experiments were done in duplicate and repeated twice.

FIG. 10. Expression of L₀ peptide in E. coli cells

FIG. 10 illustrates the expression of the fusion protein consisting of MRP1 L₀ peptide and thioredoxine (TRX) by BL21 cells, in cell lysates and various cell fractions. It can be seen that the TRX-MRP-L₀ was produced by inducing with IPTG and appeared in the soluble fraction of the cells.

FIG. 11. Amino acid sequence of MRP1 L₀ peptide

FIG. 11 shows the amino acid sequence of MRP1 L₀ peptide plus some further amino acids. The amino acid numbering corresponds to the numbering of the MRP1 sequence.

### DETAILED DESCRIPTION OF THE INVENTION

Below the invention is disclosed in more detail with reference to some examples.

In order to develop the assay method of the invention the functional properties of the approximately 280 amino acid long N-terminal region of human MRP1 were analyzed. The so called TMD₀ and L₀ regions of MRP1 (see FIG. 4A) are present in several members of the MRP family, including the SUR proteins, and partially also in CFTR, but absent in the proteins of the MDR family (MDR1, MDR3 and sPGP). By fusing the N-terminal regions of MRP1 to the human MDR1 protein, and by performing various mutation and co-expression studies, we examined the role of these regions in processing, plasma membrane targeting and transport of the multidrug resistance proteins. Proteins were expressed both in Sf9 insect cells and MDCKII mammalian cells. In the first system, due to the high levels of expression in a heterologous cell type, functional studies and convenient biochemical assays could be carried out in isolated membranes with less interference from any unrelated proteins. These assays included measurements of labeled glutathione-conjugate and leukotriene C₄ transport, as well as vanadate dependent nucleotide occlusion in isolated membrane vesicles. The polarized MDCKII cell monolayers offered the possibility to efficiently study membrane routing and transcellular transport properties of the overproduced proteins.

In order to study the role of the N-terminal regions of MRP1, inventors constructed chimeric MRP1-MDR1 proteins, in which MDR1 was extended either with the full-length N-terminal region of MRP1 (TMD₀+L₀), or with the two separate sub-components (TMD₀ or L₀) of this region. The object of these experiments was to create an assay system by which L₀function can be studied separately from the function of other parts of the MRP1-molecule, and thereby L₀-specific inhibitors can be tested. It was found, however, that though all of these proteins were properly expressed in both the Sf9 and MDCKII cells, essentially preserved the transport nucleotide occlusion and apical membrane routing features of the wild-type MDR1. A possible explanation for this unexpected result i.e. that MDR1 protein (though its core region is very similar to that of MRP1) could not be converted to MRP1 in respect of any feature, can be that specific interdomain interactions are required for the function of this region within MRP1.

Whereas the L₀ region undoubtedly has a significant role, it could not be expected from the above results that expressing it separately is useful and that said L₀ region can be studied as a separate molecule having its own functions. In spite of that, we have prepared a construct for expression of a separate L₀-molecule in Sf9 cells. It was unexpectedly found that the MRP1 core region which is unfunctional in itself, if coexpressed with a separate L₀ peptide, resulted in a functional MRP1 protein having substantially the same transport and nucleotide trapping features as the wild type protein. Thus, there is no need for a covalent linkage between the L₀ peptide and the core region of MRP1. The complex assembled from the coexpressed L₀ and ΔMRP1 had similar kinetic properties to L₀MRP1 [Bakos et al. (1998), see above].

When ΔMRP1 was co-expressed with the L₀ region in polarized MDCKII cells, we found that L₀ was routed to the lateral membrane (FIG. 6), most likely together with ΔMRP1. Similar results were obtained in co-expression experiments with the TMD₀L₀ region in which we could show a partial relocalization of ΔMRP1 and TMD₀L₀ to the plasma membrane (data not shown). Although in MDCKII cells, for technical reasons, transport by these co-produced proteins could not be measured, these results suggest that the isolated TMD₀L₀ and L₀ peptides can properly fold, attach to the cell membrane and associate with ΔMRP1. All co-expression studies in Sf9 and MDCKII cells indicate that the L₀ region is a distinct protein domain within MRP1 protein that works in specific association with the core region of MRP1.

Independently from the inventive solution it is supposed that the L₀ region contains a routing signal sufficient to induce lateral routing of the ΔMRP1 fragment. However, we can not exclude that ΔMRP1 alone is not properly folded in MDCKII cells and therefore gets stuck in the endoplasmic reticulum. Association with L₀ may result in proper folding and transport to the plasma membrane.

As described in the Examples, the L₀ peptide of the MRP1 was prepared also in bacterial expression system (in E. coli BL21 cells), separated from the core region, in soluble form as a fusion peptide linked to thioredoxine (TRX). When the lysate was added to inactive ΔMRP1 prepared in a different expression system (Sf9 cells) the activity of the latter could be restored in inside-out Sf9-vesicles. This result shows that L₀ peptide produced in a bacterial system has a typical structure ensuring capability of functioning, and further suggests that L₀ region is a distinct domain within MRP1. Using the bacterial expression system it is possible to prepare a native functional peptide easy to purify. The obtained L₀ peptide provides an opportunity to monitor interactions with this region of MRP family transporter proteins, and to develop or test specific inhibitors more effectively.

Thus, with the set of experiments disclosed above and by developing meanwhile peptide molecules, polynucleotides, vectors and host cells, actually an assay method was created for testing L₀-specific inhibitors of transporter proteins of the MRP family, comprising assaying an L₀ peptide exposed to the effect of a potential inhibitor, to determine whether at least one L₀-function was retained or at least partially lost. Preferably, in the presence and in the absence of the potential inhibitor, it is determined whether at least one L₀-function was retained or not and the results obtained in each case are compared. In a preferred embodiment L₀ peptide is expressed. If the function studied is not independent from translation, folding or processing (resulting in the final native structure), the inhibitor should be added to the system during these periods. In a further preferred embodiment, when assaying the L₀-function, the core region of an MRP-family transporter protein or its fragment capable of functioning, is also examined in respect of at least one L₀-function manifesting itself only upon contact with the core region. In a further preferred embodiment the core region or a protein comprising a functional fragment thereof is also expressed. L₀-functions, which can be preferably assayed are e.g. membrane binding, rescuing the core region of the protein to the physiological membrane compartment and/or restoring transporter protein function.

Preferably, it can be examined whether at least one L₀-function manifesting itself only upon contact with the core region can be restored or not. Such functions are e.g.: nucleotide trapping (occlusion) in membrane preparations; membrane routing, preferably to a specific membrane area of polarized cells; ATP-ase activity; membrane transport, preferably substrate uptake in inside-out vesicles; vectorial membrane transport into or from polarized cells.

The assay method can be carried out also preparing the L₀ peptide and the core region in different expression systems (e.g. in bacterial cell and insect cell, respectively), and contacting them afterwards. Then the potential inhibitor may block (if added previously) or abolish (if added later) the MRP-function which could have been or was restored.

Besides functional studies, it is expedient to study the potential inhibitor in respect of its binding to or affinity to L₀ peptide or its amphipatic helix, by using the L₀ peptide or its mutants or fragments of the invention. Technologies useful for that purpose, such as spectroscopic methods (e.g. infrared, ultraviolet or CD spectroscopic methods), sedimental measurements, measurements based on light scattering mobility etc. or nuclear magnetic resonance experiments are well known by the skilled person.

Peptides, polynucleotides, vectors and host cells disclosed according to the invention are also claimed. Some illustrative examples of these are described in the Examples section. Molecules and assay methods of the invention are useful as developing and testing drugs behaving as inhibitors and for facilitating inhibitor design. Due to the above embodiments study of the disclosed key regions of MRP-family proteins, obtaining a detailed structural information thereon and thereby preparation of molecules capable of binding with strong affinity becomes possible.

Based on the above, the claimed peptides polynucleotides, vectors and host cells can be combined into reagent kits for developing and/or testing inhibitors specific for transporter proteins, as disclosed herein.

In the experiments with Sf9 membranes we found that the isolated L₀ peptide, which was predicted to be localized in the cytoplasm, was attached to membranes and could be solubilized only by treatment with urea or detergents. This membrane interaction of L₀ did not require the presence of the ΔMRP1 protein. Computer-based secondary structure predictions indicated that the L₀ region has two alpha-helical regions, one of which (amino acids 221-233) is a characteristic amphipathic helix, conserved in all members of the MRP family (FIG. 7C). Such an amphipathic helix may be bound by hydrophobic interactions to the lipid phase of the cell membrane or with hydrophobic regions of other membrane proteins.

In order to analyze the role of the predicted amphipathic alpha-helical region within L₀, a mutant was generated in which this region was deleted. When MRP1Δ(223-232) was expressed in both Sf9 and MDCKII cells, we found that this deletion eliminated MRP1 transport function, but did not affect glycosylation or membrane routing. Moreover, when the isolated L₀Δ(223-232) was produced in Sf9 cells, the mutation abolished the hydrophobic membrane attachment of the L₀ region and eliminated any functional re-activation of the ΔMRP1 construct.

A similar result was obtained when the amphipatic helix of the MRP1 was exchanged for the L₀ region amphipathic helix of another MRP-family protein (MRP2). A chimeric L₀ obtained this way and expressed in the above-described E. coli expression system was added to ΔMRP1, but could not restore its activity. However, if only the amino acids of the supposed hydrophilic site were replaced by the corresponding amino acids of the hydrophilic site of the MRP2 L₀ amphipatic helix, transport activity was restored.

Thus, the experiments showed that the predicted and experimentally supported amphipatic helix found in the L₀ region participates in membrane binding and is absolutely necessary for maintaining the functional character of MRP1. Therefore, this region is particularly useful for testing inhibitors. It can be expected that an inhibitor blocking specifically a function of this region will be a particularly specific and effective inhibitor of the whole MRP1 molecule. Preferably, the design of the inhibitor structure can be based on the structure of this segment. This peptide segment is particularly useful in the study of MRP type proteins and thus in multidrug resistance research.

The specificity of an assayed inhibitor for the segment predicted to be an amphipatic helix can be assessed, by using the methods described above, as follows: A transporter protein or a functional transporter protein fragment comprising L₀ region (or a transporter protein the function of which was restored, e.g. by coexpression) is assayed:

a) in respect of specific membrane routing and/or glycosilation of the protein or its fragment, and

b) in respect of any other function, preferably transporting capability of the protein, membrane binding of the L₀ peptide and/or capability of the L₀ peptide to restore MRP1 function, and

if the assayed functions of a) are retained and if the assayed functions of b) are at least partially considering the inhibitor to have a selective inhibiting effect specific for the segment predicted to be an amphipatic helix. As will be understood the affinity to the helix can be preferably assayed also in this embodiment.

Further, a reagent kit can be readily provided, said kit comprising an expression victor useful for expressing a mutant L₀ region, wherein the segment of said L₀ region predicted to be an amphipatic helix or a part thereof, preferably a segment corresponding to MRP1 amino acids 221 to 232 or any part thereof is deleted or substituted.

The invention is further illustrated below by detailed experimental examples.

### EXAMPLES

1. Materials and Methods

1.1 Generation of MRP1 Variants and Recombinant Baculoviruses

In order to generate the MRP1-MDR1 chimeric constructs, the full length MDR1 cDNA was cloned into the BamHI site of Bluescript SK. The BamHI-EcoRI (nt-8-1177) fragment was removed from Bluescript-MDR1 and subcloned into M13mp18 and an XbaI site was introduced by site-directed mutagenesis at nt 4 of MDR1. The XbaI-EcoRI fragment from this construct and the EcoRI-NotI (NotI of the polycloning site) fragment from Bluescript-MDR1 were ligated together into the pVL1393 baculovirus transfer vector, yielding pVL1393MDR1. Previously, an XbaI site was introduced at nt 606 of the MRP1 cDNA in M13SBMRP1 [Bakos É. et al, (1998), see above]. To obtain TMD₀MDR1 chimeric cDNA, a DraIII-BamHI fragment (nt 87-840) was removed from the pAcSG2MRP [Bakos É. et al, (1998), see above] and replaced by the DraIII-BamHI insert of M13SBMRP1. An XbaI-XbaI fragment (nt 5-606) fragment was subcloned into the XbaI site of pVL1393MDR1. To generate the TDM₀L₀MDR1 the pAcsG2PlinkerII [Bakos É. et al, (1998), see above] was digested with NheI within the linker I preceeding the stop codon and the XbaI site at position −5. The fragment was subcloned into the XbaI site of pVL1393MDR1. To obtain L₀MDR1, linker I [Bakos É. et al, (1998), see above] was inserted into the unique BamHI site of the pAcSG2MRPlinkerII. The pAcSG2MRPlinkerII/linkerI was digested with NheI within linker I and II. The fragment was subcloned into the XbaI site of pVL1393MDR1.

The MRP1Δ(223-232) mutant was generated by overlap PCR using pJ3Ω-MRP1 (23) as the template. The following primers were used: 5′-gatgtcgacaccggcatggcgctccggggcttc-3′(primer 1; SalI site underlined), 5′-cactgccctccaggggctggcgccagaaggtgatcctcgacagg-3′(primer 2), 5′-cctgtcgaggatcaccttctggcgccagcccctggagggcagtg-3′(primer 3, encoding amino acids 216-222 and 233-239, and complementary to primer 2), 5′ctcctcattcgcatccaccttggaactctc3′(primer 4, amino acids 287-297). The upstream fragment was generated using primers 1 and 2, the downstream fragment using primers 3 and 4. Both fragments were gel purified, combined and a PCR reaction with primers 1 and 4 was performed. The amplified overlap product was digested with SalI-BamHI and the fragment was substituted for the original SalI-BamHI fragment in pJ3Ω-MRP1, resulting in pJ3Ω-MRP1(Δ223-232).

To generate the isolated L₀ the pAcSG2MRPlinkerII/linkerI construct was partially digested with NheI in linker II, and SpeI in linker I. The fragment (encoding amino acids MAL+204-281+LLA+Stop) was subcloned into the XbaI site of pVL1392. To obtain the L₀Δ(223-232) fragment, the fragment encoding amino acids 204-281 was amplified by PCR using pJ3ΩMRP1(?223-232) as the template. The forward primer used was 5′-taggctagccggcatggaccctaatccctgcccaga-3′, the reverse primer used was 5′-cggctagatctaatccttggaggagtacac-3′. The primers included a NheI site and BglII site, respectively. The PCR fragment was digested, with NheI and BglII and the fragment was cloned into pVL1393. All constructs were verified by sequence analysis.

Sf9 (Spodoptera frugiperda) cells were cultured and infected with a baculovirus as described earlier (11). Recombinant baculoviruses were generated by using the BaculoGold Transfection Kit (PharMingene).

1.2 Generation of MDCKII Cells Expressing MRP1 Variants

For expression of MRP1 variants in MDCKII cells, the L₀MDR1 chimera, ΔMRP1, and MRP1Δ(223-232) were inserted as blunt-end fragments into the retroviral vector pCMV-neo as described [Bakos É et al, (1998), see above]. For co-expression experiments the L₀ fragment was cloned into the pBabe-Puro-CMV vector (kindly provided by Dr. J. Wijnholds, The Netherlands Cancer Institute, Amsterdam, The Netherlands). This vector was generated by cloning the blunt-ended HindIII fragment from pCMV-neo (containing the CMV early promoter) into the blunt-ended BamHI site of the pBabe-puro vector [Morgenstern J. P., and Land, H. (1990) Nucleic Acids Res. 18, 3587-3596]. Cells were retrovirally-transduced as described before [Evers, R. Kool, M., van Deemter, L., Janssen, H., Calafat, J., Oomen, L. C., Paulusma, C. C., Oude Elferink R. P., Baas, F., Schinkel, A. H., and Borst P. (1998) J. Clin Invest. 101, 1310-1319]. MDCKII cells stably transduced with the pBabe-Puro or pCMV-neo vector were selected for 4-6 days in medium containing puromycin (3 μg/ml) or for 7 days in medium containing G-418 (800 μg/ml), respectively.

1.3 Expression of MRP1 L₀ Peptide in E. coli Cells

In order to produce a fusion protein of L₀ peptide with thioredoxin a fragment coding amino acids 204 to 281 of MRP1 was PCR-amplified by using pVL1393-L0 as a template. The sequence of the “forward” primer ‘a’ was 5′-attggtacccgaccctaatccctgg-3′, and the sequence of the “reverse” primer ‘d’ was 5′-aagaagcttaatccttggaggagtacac-3′. The primers contained a KpnI and a HindIII endonuclease restriction site, respectively. The PCR fragment was digested by enzymes KpnI and HindIII and subcloned to the KpnI-HindIII site of the pCLTRX vector (Crosslink), resulting in pCLTRX-L0.

The MRP1-MRP2 chimeric L₀, comprising the amphipatic helix of MRP2, was prepared by the overlap PCR method. As a template pVL1393-L0 was used. The 5′ fragment was prepared by using forward primer ‘a’ and reverse primer ‘b’ (5′-teaga atgat gctgt catac cagct gtagg tgatc gtcga cagg-3′). The complementer of primer ‘b’ codes for amino acids 216 to 220 of MRP1 and amino acid 206 to 214 of MRP2. The 3′ fragment was prepared by using forward primer ‘c’ (5′-tgaca gcatc attct gaaag gctac aagca gcccc tggag ggg-3′) and reverse primer ‘d’. Primer ‘c’ codes for amino acids 210 to 218 of MRP2 and amino acids 234 to 238 of MRP1. Fragments were isolated and pooled then a PCR was carried out by primers ‘a’ and ‘d’. The obtained product was digested by KpnI and HindIII and cloned to pCLTRX vector.

The obtained pCLTRX-L0 vector was cloned to E. coli BL21 (DE3) cells, which in turn were grown in TYBG antibiotic broth (10 g tryptone, 10 g yeast extract, 4 g glucose, pH 7.2, 100 μg/ml ampicillin, 25 μg/ml kanamycine) at 37° C. When OD(600) exceeded 1.1 cells were pelleted by centrifugation, suspended in M9 medium, the incubated at 28° C. for 30 min. Afterwards, expression was induced by IPTG for 2 hours. When the induction was finished, cells were pelleted by centrifugation and the pellet was washed with 20% sacharose, then suspended in a solution comprising 20% agarose, 10 mM Tris and 0.1 μg/ml lysosyme (pH 7.2) and incubated for 30 min at 4° C. then sonicated. The lysed cells were pelleted by centrifugation (20000 g, 20 min). The so obtained lysate was used in the further experiments, for studying the restoration of the transport activity.

1.4 Membrane Preparation and Immunoblotting

The virus-infected Sf9 cells were harvested, their membranes isolated and stored, and the membrane protein concentrations determined as described [Sarkadi B., (1992) see above]. Immunoblotting detection of human MRP1 with the R1 and M6 monoclonal antibodies, and detection of protein-antibody interactions using the enhanced chemiluminescence technique were as described previously [Bakos É, et al., (1996), see above].

1.5 Tracer Uptake in Membrane Vesicles and Vanadate-Dependent Nucleotide Trapping

(³H)LTC₄ (135 Ci/mmol) was obtained from DuPont NEN, (³H)NEM-GS was prepared from (³H)NEM (60 Ci/mmol, NEN Life Science Products) by mixing the isotope in 10 mM Tris-HCl (pH 7.0) with freshly dissolved GSH in a 1:1.1 molar ratio. For (₃H)NEM-GS and (³H)LTC₄ transport measurement, isolated membranes (200 μg of protein) were incubated in the presence of 4 mM ATP or AMP in 500 μl of transport buffer (10 mM MgCl₂, −40 mM MOPS-Tris, pH 7.0, 50 mM KCl) at 23° C. (LTC₄) or at 37° C. (NEM-GS). 100 μl aliquots of this suspension were added to 1 ml of ice-cold transport buffer at the time points indicated and subsequently filtered rapidly though nitrocellulose membranes (0.25 μm pore size). The filters were washed twice with 0.5 ml of cold transport buffer, and filter-bound radioactivity was measured in scintillation fluid (Optifluor, Packard). ATP-dependent transport was calculated by subtracting the activity values obtained with AMP from those in the presence of ATP. Vanadate-dependent nucleotide trapping was measured basically as described elsewhere [SzabóK. et al, (1998) see above].

1.6 Confocal Laser Scanning Microscopy

For immunofluorescence experiments cells were grown on microporous polycarbonate filters (3 μm pore size, 24.5 mm diameter, Transwell™ 3414; Costar Corp., Cambridge, Mass.) at a density of 4×10⁵ cells/wells for 3 days. Antibody incubations with mAbs M6 and R1 were as described before [Bakos É et al., (1998) see above]. Incubations with the anti-MDR1 mAb MRK16 was performed as above with the modification that cells were not permeabilized with Triton X-100. FITC-labeled secondary antibodies were from Zymed Laboratories, Inc. and from Roche diagnostics.

1.7 Transport Assay with MDCKII Cells

Transport assays were carried out as described [Evers et al, (1996) and (1998), see above with the modification that in [¹⁴C]DNP-GS transport experiments, Hanks' buffered saline solution [Evers et al., (1998) see above] was used instead of Dulbeccos's modified Eagle's medium.

1-chloro-2,4-dinitro[¹⁴C]benzene(10Ci/mol), and [³H]vinblastine(9.4 Ci/mmol) were obtained from Amersham Pharmacia Biotech.

2.Chimeric MRP1-MDR1 Variants, Their Expression and Functions

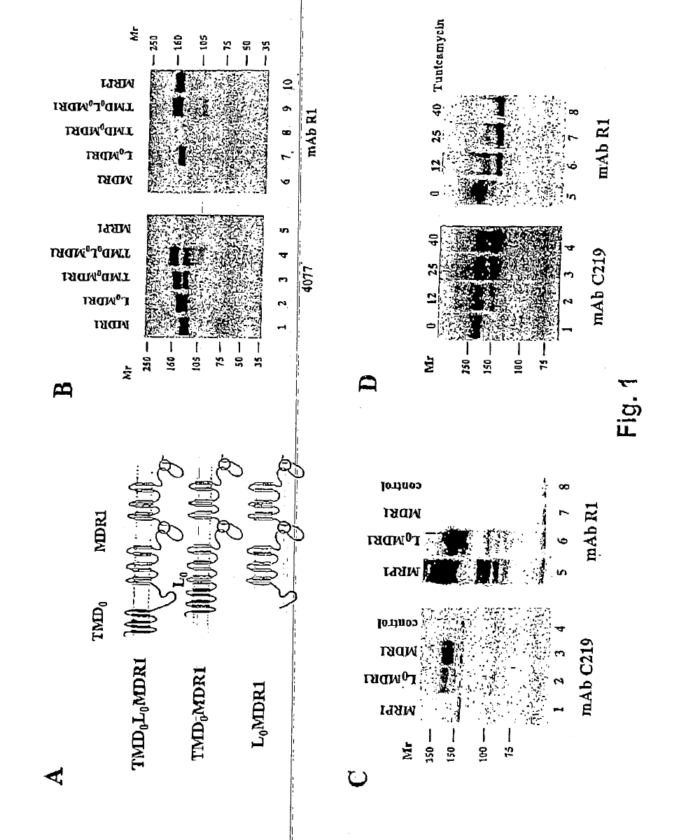

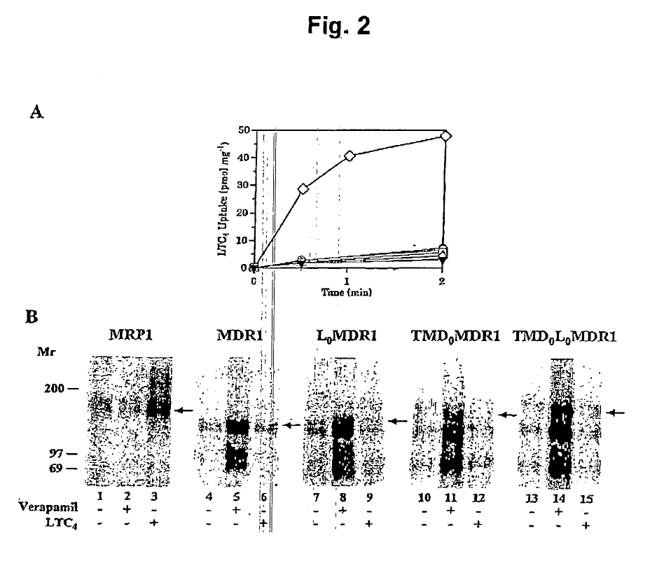

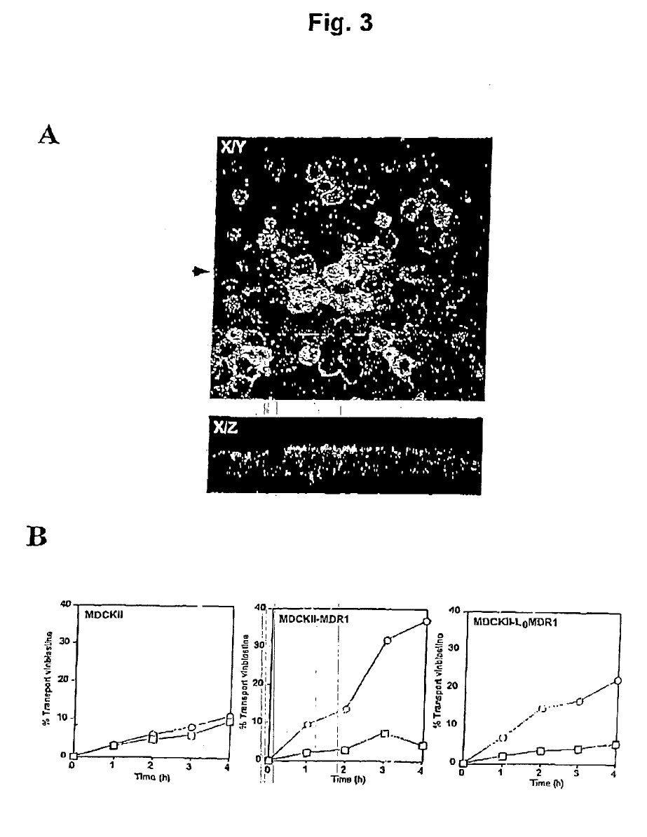

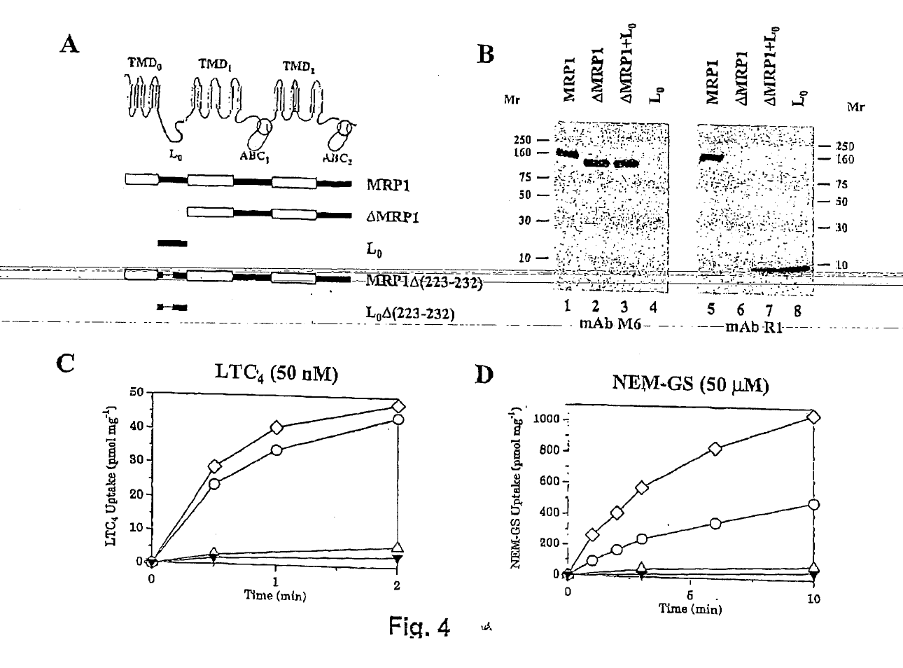

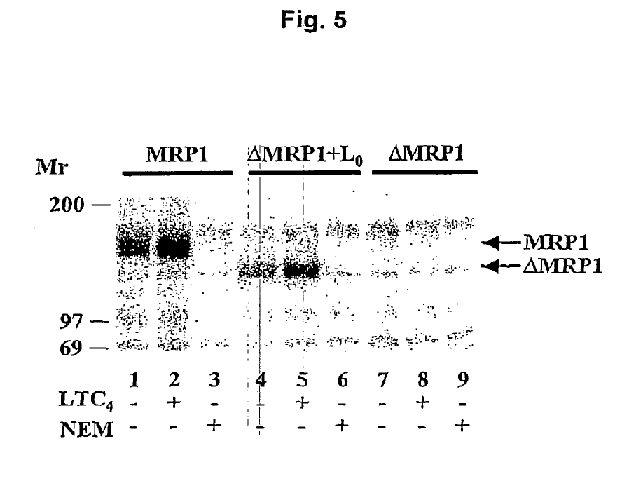

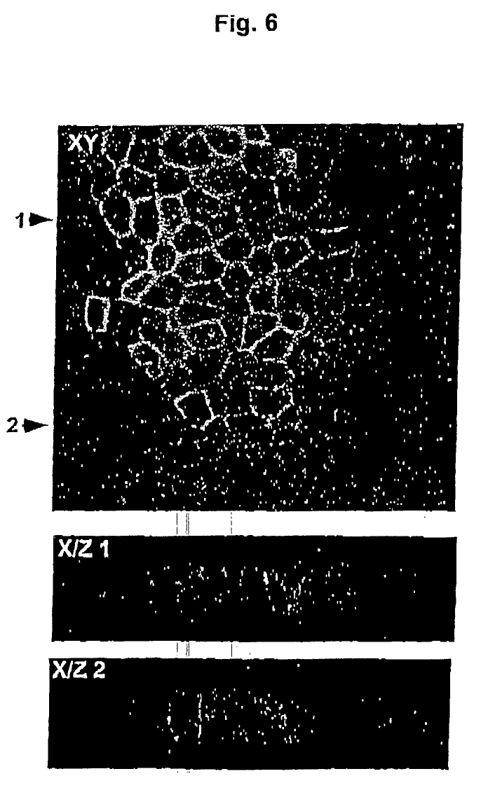

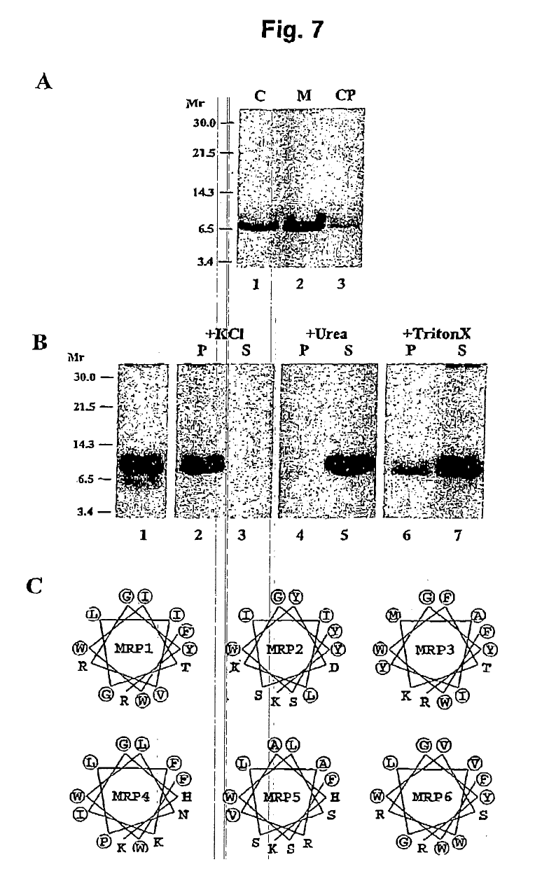

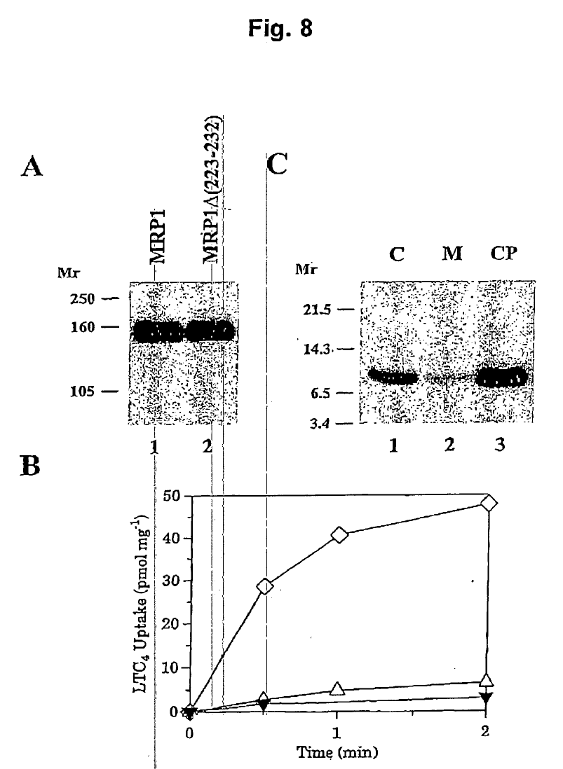

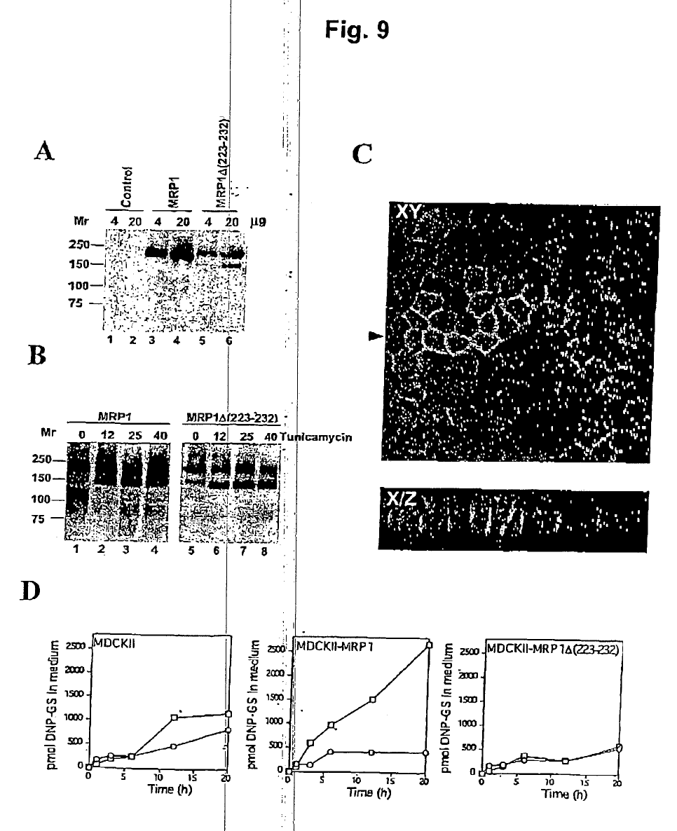

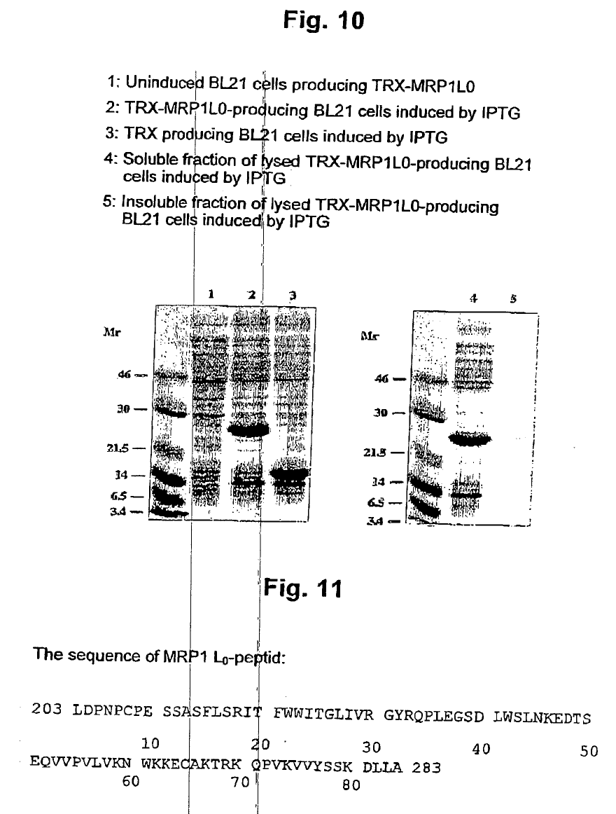

We have inserted cDNAs into baculovirus and retroviral vectors to obtain the following molecules: (i) L₀ of MRP1 (amino acids 204-281), covalently coupled to the N-terminus of MDR1 (L₀MDR1); (ii) TMD₀ of MRP1 (amino acids 1-203), covalently coupled to the N-terminus of MdR1 (TMD₀MDR1) and (iii) the full N-terminal region of MRP1 (TMD₀L₀, amino acids 1-281), covalently coupled to the N-terminus of MDR1 (TMD₀L₀MDR1; see FIG. 1A). The MRP1-MDR1 chimeras were expressed in baculovirus-infected Sf9 cells and in retrovirus-infected MDCKII cells. For this latter system we only present the data for the L₀MDR1 chimera, since we focused on the development of an assay method for detecting L₀-specific inhibitors.

The protein levels and the transport properties of the various mutants were studied in intact cells and in isolated membrane preparations. FIG. 1 (panels B, C, and D) presents the imnunoblots of the proteins detectable in membrane preparations of Sf9 cells and in retrovirally-transduced MDCKII cells, respectively (see example 1.4). The epitope for mAb R1 is within the L₀ region [amino acids 238-247; [Hipfner, D. R, Gao, m, Scheffer, G., Scheper, R. J., Deeley, R. G., and Cole, S. C. P. (1998) Br. Journal of Cancer 78, 1134-1140], while those for the polyclonal Ab 4077 and mAb C219 are within MDR1 Pgp. We obtained significant expression of the chimeric MRP1-MDR1 proteins, commensurable to those of the wild-type molecules in the same expression systems. A small amount of partial proteolytic cleavage was apparent in proteins produced in the baculovirus expression system (FIG. 1B), at sites close to the artificially constructed joining regions. However, the majority of the produced proteins was found at molecular mass values corresponding to the full length chimeras. In MDCKII cells the L₀MDR1 protein, like MDR1, was glycosylated and treatment of the cells with tumicamycin abolished this glycosylation (FIG. 1D).

In order to examine the functional character of the chimeric proteins, we first measured ATP-dependent LTC₄ and NEM-GS transport in isolated Sf9 cell membrane vesicles (see, example 1.5). Despite the appreciable amounts of protein detectable in the vesicles, we did not find an MRP1-like GS-conjugate transport activity exceeding that observed in the control, β-galactosidase containing membrane vesicles (FIG. 2A).

In the absence of any MRP1-like transport activity it was important to determine if the MRP1-MDR1 chimeras retained MDR1-like hydrophobic drug transport capacity. Since direct vesicular port measurements with hydrophobic MDR1 substrates are difficult to perform, we examined the substrate-simulated ATPase and nucleotide trapping activity of the MRP1-MDR1 chimeric proteins in isolated Sf9 membranes.

In all membranes containing the chimera MRP1-MDR1 proteins we found a significant verapamil-stimulated ATPase activity (not shown). However, this activity may be the consequence of a partial proteolysis of the chimeras (see FIG. 1B), yielding full-length MDR1, thus nucleotide trapping was considered more informative in this regard. Covalent photoaffinty labeling of the proteins was performed with Mg-8-azido-[α-³²P)-ATP in the presence of vanadate as a trapping agent (set Materials and Methods). It has been known that the MDR1 protein is capable of nucleotide trapping when stimulated by a hydrophobic compound (e.g. verapamil) and that it is not affected by LTC₄. This activity in MRP1 is significantly simulated by LTC₄, but not by verapamil [Szabó, K et al and Bakos, É. et al, (1998), see above].

As shown in FIG. 2B, the nucleotide trapping of the MRP1-MDR1 chimeras was easily measurable and was vanadate-dependent. Stimulation by verapamil was observed both in the protein bands representing the full-length MRP1-MDR1 chimeras, as well as in the proteolytic fragments representing the MDR1 core. In all cases a full inhibition of nucleotide trapping was obtained by pretreatment with 1 mM N-ethymaleimide (data not shown ). These functional assays demonstrate that the MRP1-MDR1 chimeras retained their MDR1-like nucleotide trapping function.

In the following experiments, we examined the membrane localization and transport function of the L₀MDR1 chimeric protein in retrovirally transduced MDCKII cell monolayers by Confocal Laser Scanning Microscopy (CLSM). L₀MDR1 was detected using mAb MRK16, an antibody recognizing a conformational epitope on the extracellular loops of MDR1 Pgp [Hamada, H., and Tsuruo, T. (1986) Proc. Natl. Acad. Sci. USA 83, Cancer Res. 7735-7789). As shown in FIG. 3A, L₀MDR1 was predominantly localized in the apical membrane of MDCKII cell monolayers. In accordance with an apical localization, the MDCKII-L₀MDR1 and MDCKII-MDR1 cells showed an increased apical transport of vinblastine (FIG. 3B). The vectorial transport activity of L₀MDR1 correlated with its protein levels as compared to that of MDR1 (FIG. 1C). We found no increased transport of organic anions, more closely DNP-GS, by the MDCKII-L₀MDR1 cells (data not shown).

The combination of these results clearly indicates that the MRP1-MDR1 chimeras were produced in both insect and mammalian cells and that the chimeric proteins retained the features of the MDR1 protein. Since L₀, when coupled to MDR1 protein, does not result in any change in functions, its functional behaviour can not be studied in the system described above. Therefore, no assay system, suitable for testing of L₀-specific inhibitors, could be created using chimeric constructions disclosed in this example.

3. Mutant MRP1 Variants, Their Expression and Functions

In order to analyse the specific role of the N-terminal linker region of MRP1 in more detail, we have expressed the following mutant MRP1 constructs in both Sf9 and MDCKII cells: (i) An N-terminally truncated MRP1 (amino acids 281-1531), containing the two ABC units and the 2×6 TM (transmembrane) helices [ΔMRP1; Bakos É et al, (1998), see above]; (ii) amino acids 204-281, representing the N-terminal linker region of MRP1 (L₀), (FIG. 4.A).

In the experiments below the existence of MRP1 functions was examined for each function, in case of ΔMRP1 and co-expressing ΔMRP1 and L₀ peptide, and analysed the behaviour of L₀ in itself and what functions can be restored by it.

3.1 Expression in Sf9 Cells

FIG. 4B demonstrates the immunoblot detection of the ΔMRP1 and L₀ variants in Sf9 cells by the anti-MRP1 monoclonal antibodies R1 and M6. The epitope for mAb M6 is at the C-terminus of MRP1 [amino acids 1511-1520; Hipfner et al., (1998) see above]. For all these molecules we obtained appreciable expression, similar to that of wild-type MRP1 (FIG. 4B). It is important to note (see also below) that, like wild-type MRP1 and ΔMRP1, the L₀ polypeptide was found to be almost entirely present in the membrane fraction of the cell lysates. The amount of this membrane-bound L₀ was independent of the presence or absence of ΔMRP1 in the Sf9 cell membranes. Slight variations in the expression levels of these mutant proteins between various membrane preparations were still observed and therefore the functional data were corrected for the specific protein expression levels (see below).

3.2 Transport of MRP1 Substrates [LTC₄ and NEM-GS Muller, M. et al, (1994), Bakos É. et al, (1998), See Above].

In order to correctly determine the transport function of the different MRP1 proteins, we estimated the relative amount of transport-competent inside-out vesicles in the Sf9 membrane preparations [Bakos É. et al, (1998), see above], and corrected the transport rates according to the relative MRP1 expression levels, as determined by immunoblotting.

FIG. 4, panels C and D, shows the time-dependent uptake of [³H]LTC₄ (50 nM) and [³H]NEM-GS (50 μM) in Sf9 membrane vesicles containing MRP1, ΔMRP1, or the co-expressed L₀ and ΔMRP1. Full-length MRP1 produced an ATP-dependent increase in the vesicular radioactivity which for LTC₄ was linear for about 30 seconds at 23° C. and was linear for NEM-GS for about 3 minutes at 37° C. The initial rate of ATP-dependent LTC₄ transport by the MRP1 containing membranes was about 60 pmol mg⁻¹min⁻¹ while that of NEM-GS transport was about 250 pmol mg⁻¹ under these conditions. As shown previously (20) rapid washing of the vesicles with cold substrate-containing solutions did not alter the vesicular LTC₄ and NEM-GS levels, while the addition of concentrated sucrose (1 M) or low concentrations of Triton X-100 (0.02%) to the media eliminated ATP-dependent tracer accumulation.

As demonstrated in FIGS. 4C and D, we found that the expression of ΔMRP1 did not result in any transport activity (as compared to transport measured in the control, β-galactosidase expressing vesicles). However, the co-expression of ΔMRP1 and the L₀ region resulted in significant uptake of LTC₄ (at 50 nM about 90% of MRP1) and NEM-GS (at 50 μM NEM-GS about 40% of MRP1). Again, in all these cases intravesicular tracer uptake was eliminated by the addition of Triton X-100 or 1 M sucrose.

Examination of the kinetic parameters for the transport of LTC₄ by the co-expressed ΔMRP1 and L₀ showed that the Km values were similar as for wild-type MRP1 (about 140 nM). However, for transport of NEM-GS the Km of wild-type MRP1 was 150 μM, while that of the co-expressed ΔMRP1+L₀ was about 400 μM. The maximum transport rate in this latter case was approaching the level observed for MRP1 (data not shown).

Thus, co-expression of L₀ region and ΔMRP1 provides a possibility to determine an L₀-function separately, namely that L₀ is capable of restoring ΔMRP1 activity n respect of MRP1 substrates. Thus, the system unexpectedly proved to be promising in testing L₀-specific inhibitors.

3.3 Vanadate Sensitive Nucleotide Trapping in Sf9 Cell Membranes

Next, we examined substrate-stimulated, vanadate sensitive nucleotide trapping in Sf9 cell membranes containing the various MRP1 variants (FIG. 5). As reported before [Bakos É. et at, (1998), see above], ΔMRP1 did not show any nucleotide trapping. However, as shown in FIG. 5, when the L₀ peptide was co-expressed with the ΔMRP1 region, the full activity was restored and a stimulation of the nucleotide trapping by LTC₄ was observed, similar to that found for wild-type MRP1. Again, in all cases a full inhibition of nucleotide trapping was obtained by pretreatment with 1 mM N-ethylmaleimide.

Thus, it was found again that the L₀-function restoring MRP1 activity can be tested and thereby a further method, suitable for assessing effectiveness of an L₀-specific inhibitor, was provided.

3.4 Rescue of ΔMRP1 to the Plasma Membrane

It was shown previously that ΔMRP1 in MDCKII cells is localized in an intracellular compartment, probably the endoplasmic reticulum [Bakos É. et al, (1998), see above]. To investigate whether the L₀ peptide could rescue ΔMRP1 to the plasma membrane, we transduced MDCKII-ΔMRP1 cells with the pBabe-Puro-CMV-L₀ construct (Example 1.2). Upon selection with puromycin the localization of L₀ was determined using mAb R1. In a pool of puromycin resistant cells clear lateral plasma membrane staining was observed in small groups of cells, although some intracellular staining was also present in these cells (FIG. 6). As only a low percentage of cells showed staining with mAb R1, it was impossible to isolate single clones. Therefore, immunoblotting and transepithelial transport measurements could not be preformed. No signal was obtained after infection of wild-type cells with the L₀ containing construct, probably due to rapid degradation of this small protein in the absence of ΔMRP1.

The studies documented above strongly suggest the isolated L₀ polypeptide is able to associate with ΔMRP1 and that as a consequence the protein regains its function and routes to the lateral membrane. An L₀-specific inhibitor will most probably inhibit this function, too.

3.5 Membrane Association of L₀ Peptide

As mentioned above (see FIG. 4B), a significant amount of the L₀ protein produced in Sf9 cells was found to be associated with cellular membranes. FIG. 7A documents that in the Sf9 cell lysates, most of the L₀ protein was found in the membrane fraction and that only a small fraction was in the cytoplasm. Washing the Sf9 cell membranes with 1 M KCl did not change this membrane-bound localization of the L₀ peptide. However, washing the membranes with 5 M urea or 1% Triton X-100 completely solubilized the L₀ peptide (FIG. 7B). Co-expression of ΔMRP1 in the same cell did not affect the binding of L₀ to the membrane or sensitivity towards treatment with urea or Triton X-100. Treatment of membranes with urea had no effect on the membrane localization of MRP1 or ΔMRP1, and even treatment with 1% Triton X-100 resulted only in a 20-40% solubilization of these membrane-spanning proteins (data not shown).

Thus, membrane association of L₀ peptide can be studied separately and can be considered as a L₀-function which can be inhibited.

It follows from the above that functions of L₀ peptide can be assayed in a system comprising one or more methods disclosed above. Expectedly it is true for each transporter protein of the MRP-family having an L₀ region homologous to or functionally corresponding to the L₀ region of MRP1.

3.6 Expression of L₀ Peptide in a Bacterial Expression System

L₀ peptide was expressed in a soluble form, as a fusion protein, using an expression vector operable in BL21 bacterial cells (Promega). The MRP1 L₀ region cloned into pCLTRX vector (Crosslink) comprising a coding sequence for thioredoxin (TRX) fusion protein and obtained pCLTRX-L₀. Using this vector protein was produced in BL21 cells (see MATERIALS AND METHODS). Cell were lysed with lysosyme and the soluble fraction of them was separated by centrifugation. The photo of the gel stained by Coomassie shows that TRX-L₀ was produced in a soluble form (FIG. 10).

The effect of TRX-L₀ to the transport activity of ΔMRP1 was assayed, too. Lysate of cells containing TRX-L₀ was added to membrane vesicles containing ΔMRP1, then ATP dependent vesicular LTC₄ uptake was measured. The transport activity of the inactive ΔMRP1 was restored.

3.7 The Study of the Conservative, Hydrophobic, Membrane Biding Segment of the L₀ Peptide

The salt-insensitive, but denaturing agent- or detergent-sensitive membrane attachment of the L₀ region raised the possibility of the presence of a membrane-attaching amphipathic region (e.g helix) within L₀. Indeed, as shown in FIG. 7C, secondary structure predictions indicated the presence of such a conserved amphipathic helix in all currently known MRP homologs (such a helix is also present in the MRP-related CFTR and SUR proteins; data not shown).

In order to examine the role of this amphipathic helix within the L₀ region, we constructed a 10 amino acid deletion (Δ223-232) within this predicted helix, both in the context of the isolated L₀ fragment and in the full-length MRP1 molecule. FIG. 8A demonstrates that MRP1Δ(223-232) was produced in Sf9 cells in similar quantities as the wild-type MRP1 protein. FIG. 8B shows LTC₄ transport experiments in isolated Sf9 cells expressing MRP1Δ(223-232), and FIG. 8C shows the membrane interaction of L₀(223-232). In contrast to wild-type MRP1, MRP1Δ(223-232) had no appreciable transport activity and the L₀Δ(223-232) did not attach to the cell membrane. When the L₀Δ(223-232) was co-expressed with ΔMRP1 or when a concentrated supernatant containing L₀Δ(223-232) was added to isolated ΔMRP1 containing-membranes, no transport activity for LTC₄ was observed (data not shown).

FIG. 9 shows the expression, localization and function of the MRP1Δ(223-232) protein produced in MDCKII monolayers. As shown in FIG. 9A, the MRP1Δ(223-232) protein was efficiently expressed and similar to MRP1, Was almost completely glycosylated in these cells. Tunicamycin treatment resulted in the loss of glycosylation both in the MRP1 and the MRP1Δ(223-232) expressing cells (FIG. 9B). Similar to wild-type MRP1 [Bakos É. et al, (1998), see above], MRP1Δ(223-232) was localized in the lateral membrane in MDCKII cells (FIG. 9C). However, when the transport activities of MRP1 and MRP1Δ(223-232) were compared in polarised cell monolayers, there was no detectable increase in the export of DNP-GS by the MRP1Δ(223-232) protein (FIG. 9D) relative to the baseline export activity of the cells. In a further set of experiments the amphipatic helix of MRP1 was exchanged with the L₀ region amphipatic helix of another member of the MRP-family (MRP2). When the so obtained chimeric L₀ was added to ΔMRP1, it could not restore ΔMRP1 activity.

Expression systems and constructs desclosed in the description are illustrative and non-limiting.

The segment of L₀ peptide predicted to be an amphipatic helix and having key importance, preferably the segment corresponding to amino acids 221 to 232, is particularly suitable for designing inhibitors specific to said segment. Using the constructs disclosed above and by assaying appropriate L₀-functions any potential inhibitor can be tested also for its specificity for this conservative region within the L₀ being particularly important for membrane binding. If such a preferred inhibitor is to be identified, a mutant construct can be created from which this region so important in membrane binding is deleted or in which is inactivated. Such a mutant may have certain functions which are different from those taught herein.

It is to be understood that by deleting or substituting a shorter or a longer amino acid segment a molecule mutated on substantially the same principle, can be prepared, which can be used for testing or developing inhibitors, for finding narrower key segments or key amino acids of proteins of the MRP-family, for understanding structural correlations and thereby disclosing functions. Informations obtained this way can be used in inhibitor or drug design.

Since it is known that MRP1 plays an important role in resistance of human tumors against clinical chemotherapy [Hipfner, D. R., Deeley, R. D, ad Cole, S. P. C. (1999) Biochim Biophys Acta 1461, 359-376], the specific and effective assay method, molecules and host cells of the invention may have a significant part in research of transporter proteins of the MRP-family, testing and designing inhibitors of them and thereby combatting multidrug resistance.

1. An assay method for testing L₀-specific inhibitors of transporter proteins of the MRP family, comprising assaying an L₀ peptide exposed to the effect of a potential inhibitor, to determine whether at least one L₀-function was retained or at least partially lost.

2. A method of claim 1 comprising expressing the L₀ peptide and optionally exposing the L₀ peptide to the effect of the potential inhibitor before the final formation of its native structure.

3. A method of any of claims 1 or 2 wherein assaying the L₀ peptide comprises the steps of using a deletion mutant protein prepared by expression or its functional fragment, said protein or fragment comprising an MRP-family transporter protein core region; and assaying at least one L₀-function manifesting itself only upon contact with the core region.

4. A method of claim 3 comprising expressing; or optionally coexpressing, both the L₀ peptide and the core region or its functional fragment.

5. A method of any of clams 3 or 4 comprising assaying L₀ peptide to determine whether at least one of the following functions was retained: membrane binding, rescuing the core region of the protein to the physiological membrane compartment and/or restoring transporter protein function.

6. A method of any of claims 3 to 5 wherein the at least one L₀-function manifesting itself only upon contact with the core region is selected from the following: nucleotide trapping in membrane preparations; membrane routing, preferably to a specific membrane area of polarised cells; ATP-ase activity; membrane transport, preferably substrate uptake in inside-out vesicles; vectorial membrane transport into or from polarised cells.

7. An assay method for screening potential inhibitors a transporter protein of the MRP family said inhibitor being specific for a segment of the L₀ region predicted to be an amphipatic helix comprising determining whether the potential inhibitor exhibits affinity for the segment predicted to be an amphipatic helix.

8. An assay method for testing inhibitors of MRP family transporter proteins specific for an L₀ region segment predicted to be an amphipatic helix, comprising assaying a transporter protein of the MRP family having exposed to the effect of a potential inhibitor; a functional transporter protein fragment comprising L₀ region; or an L₀ peptide and a protein consisting of a transporter protein core region (or a functional fragment thereof) said peptide and said protein being not linked covalently;
a) in respect of specific membrane routing and/or glycosilation of the protein or its fragment, and
b) in respect of any other function, preferably transporting capability of the protein, membrane binding of the L₀ peptide and/or MRP1 function restoring capability of the, L₀ peptide, and
if the assayed functions of a) are retained and if the assayed functions of b) are at least partially lost considering the result as the selective inhibiting effect of the inhibitor specific for the segment predicted to be an amphipatic helix.

9. L₀ peptide or a mutant or homologue thereof retaining at least partly at least one L₀-function, for use in testing, designing or developing L₀-specific inhibitors of transporter proteins of the MRP family, in multidrug resistance research or as a validated drug target.

10. L₀ peptide or its mutant of claim 9 comprising amino acids 221 to 227 of MRP1 L₀ peptide.

11. Mutant L₀ peptide carrying a mutation in the peptide segment predicted to be an amphipatic helix, preferably in the segment consisting of amino acids 221 to 227, said mutation being preferably a deletion or a substitution.

12. A polynucleotide comprising a sequence coding L₀ region of a transporter protein of the MRP family for use in expressing L₀ peptide.

13. A polynucleotide of claim 12 wherein said polynucleotide is an expression vector, preferably an expression vector capable of functioning in a bacterial, an insect and/or a mammalian cell.

14. A host cell carrying a vector of claim 13.

15. A reagent kit for developing or testing specific inhibitors of transporter proteins of the MRP family or for multidrug resistance research, said kit comprising
a peptide of any of claims 9 to 11, a polynucleotide of any of claims 12 to 13 or a host cell of claim 14,
and optionally further comprising
an expression vector suitable to express the transporter protein or a functional mutant thereof, preferably a deletion mutant substantially consisting of the core region of the transporter protein,
host cells, preferebly bacterial, insect and/or mammalian cells, in which the expression vector is operable, and
reagents and buffers for the expression and/or assaying functions of the expressed L₀ peptide.

16. A reagent kit according to claim 15, wherein the vector is a vector operable in E. coli, Sf9 and/or MDCKII cells and the host cells are E. coli, Sf9 and/or MDCKII cells.

17. A drug behaving as an L₀-specific inhibitor of a protein of the MRP family, if assayed by a method of any of claims 1 to 8.

18. A drug according to claim 17 behaving as an inhibitor, wherein said drug is capable to effect due to a contact with an L₀ region segment predicted to be an amphipatic helix or a part thereof.
